(19) United States
(12) Patent Application Publication
(10) Pub. No.: US 2016/0353999 A1
(43) Pub. Date: Dec. 8, 2016

(54) SPHYGMOMANOMETER

(71) Applicant: BOE TECHNOLOGY GROUP CO., LTD., Beijing (CN)

(72) Inventor: Zuo Yuan, Beijing (CN)

(73) Assignee: BOE TECHNOLOGY GROUP CO., LTD., Beijing (CN)

(21) Appl. No.: 15/026,380

(22) PCT Filed: Nov. 11, 2015
(86) PCT No.: PCT/CN2015/094323

§ 371 Date: Mar. 31, 2016

(30) Foreign Application Priority Data
May 26, 2015 (CN) 201510275205.5

(51) Int. Cl. A61B 5/021; A61B 5/00

(52) U.S. Cl. CPC A61B 5/02125; A61B 5/742; A61B 5/7275; A61B 5/002; A61B 5/6831

(57) ABSTRACT

A sphygmomanometer is provided, including a housing (1); a circuit board (2) and a power supply (3) that are disposed within the housing (1); and a first electrode (5), a second electrode (6) and a photoelectric sensor (7) that are electrically connected to the circuit board (2), respectively; wherein the first electrode (5), the second electrode (6) and the photoelectric sensor (7) are embedded at a surface of the housing (1).

### FIELD

Embodiments of the present invention refer to a sphygmomanometer.

### BACKGROUND

Blood pressure is a very important physiological parameter of human body. Regular measure of blood pressure is advantageous for earlier detection and diagnosis of the variety of hypertension disease so as to propose appropriate therapeutic recommendation thereto.

At present, existing devices for measuring blood pressure usually comprise standard mercury sphygmomanometer and electronic sphygmomanometer. Practical operation of the standard mercury sphygmomanometer requires a user to possess certain specialized knowledge, which results in relatively narrower application range. The electronic sphygmomanometer doesn't require the user to possess much specialized knowledge for practical operation, and hence is relatively broader in application range. However, both of the standard mercury sphygmomanometer and the electronic sphygmomanometer require the user to wear a wrist strap belt, with relatively complicated operation process; moreover, the standard mercury sphygmomanometer and the electronic sphygmomanometer both have relatively larger size, which makes the miniaturization and portability of the sphygmomanometer unachievable and cannot satisfy demand of user who requires for measurement of blood pressure whenever and wherever possible.

Therefore, the present filed always has a demand for sphygmomanometers with simple operation and small size for portability.

### SUMMARY

To this end, embodiments of the present invention provide a sphygmomanometer with advantageous such as simple operation and small size for portability.

Embodiments of the present invention provide a sphygmomanometer, comprising a housing; a circuit board and a power supply that are disposed within the housing; and a first electrode, a second electrode and a photoelectric sensor that are electrically connected to the circuit board, respectively; wherein the first electrode, the second electrode and the photoelectric sensor are embedded at a surface of the housing.

In some embodiments, the second electrode and the photoelectric sensor are embedded at the same side of the housing.

In some embodiments, the first electrode and the second electrode are embedded at two opposite sides of the housing, respectively.

In some embodiments, a surface of the first electrode exposed from the housing has a length of 15 mm to 25 mm, the surface of the first electrode exposed from the housing has a width of 8 mm to 12 mm, and the first electrode has a thickness of 1 mm to 1.5 mm.

In some embodiments, a surface of the second electrode exposed from the housing has a length of 15 mm to 25 mm; the surface of the second electrode exposed from the housing has a width of 8 mm to 12 mm; and the second electrode has a thickness of 1 mm to 1.5 mm.

In some embodiments, a surface of the photoelectric sensor exposed from the housing has a length of 5 mm to 6 mm; the surface of the photoelectric sensor exposed from the housing has a width of 2 mm to 2.5 mm; and the photoelectric sensor has a thickness of 1 mm to 1.5 mm.

In some embodiments, the sphygmomanometer further comprises a third electrode electrically connected to the circuit board; wherein the third electrode and the second electrode are embedded at the same side of the housing.

In some embodiments, a surface of the third electrode exposed from the housing has a length of 15 mm to 25 mm; the surface of the third electrode exposed from the housing has a width of 8 mm to 12 mm; and the third electrode has a thickness of 1 mm to 1.5 mm.

In some embodiments, the sphygmomanometer further comprises a wrist strap belt connected to the housing.

In some embodiments, the sphygmomanometer further comprises at least one selected from the group consisting of a Bluetooth device, and a display embedded at a surface of the housing.

During practical operation of the sphygmomanometer as provided by embodiments of the present invention, for measuring the blood pressure, it only requires a user to contact the first electrode and the second electrode with his/her two hands respectively while contacting the photoelectric sensor with his/her one hand; as compared with the existing sphygmomanometer, it's simpler in operation without the need of wearing a wrist strap belt; moreover, the first electrode, the second electrode and the photoelectric sensor are all embedded at the surface of the housing so that it not only doesn't affect the acquisition of electrocardiosignal of the first electrode and the second electrode and the acquisition of pulse wave signal of the photoelectric sensor, but also achieves miniaturization and portability of the sphygmomanometer by integrating the first electrode, the second electrode and the photoelectric sensor within the housing, thereby satisfying the demand of user who requires for measurement of blood pressure whenever and wherever possible.

### BRIEF DESCRIPTION OF THE DRAWINGS

In order to clearly illustrate the technical solution of the embodiments of the present invention, the drawings of the embodiments will be briefly described in the following. Obviously, the drawings described as below merely refer to some embodiments of the present invention without limiting the present invention thereto.

FIGS. 1-4 are schematically structural diagrams illustrating a sphygmomanometer as provided by embodiments of the present invention.

FIG. 5 is an oscillogram of electrocardiosignal and pulse wave signal as acquired by the sphygmomanometer as provided by embodiments of the present invention.

### DETAILED DESCRIPTION

In order to make objects, technical solutions and advantages of the embodiments of the present invention apparent, the technical solutions of the embodiment will be described in a clearly and fully understandable way in connection with the drawings related to the embodiments of the present invention. It is obvious that the described embodiments are just a part but not all of the embodiments of the present invention. Based on the described embodiments herein, those skilled in the art can obtain other embodiment(s), without any inventive work, which should be within the scope of the present invention.

Hereafter, particular implementations of the sphygmomanometer as provided by embodiments of the present invention are further described in more details with reference to the appending drawings.

A shape or a dimension of respective components in the drawings is not intended to reflect an actual scale thereof but only to illustratively explain contents of the present invention.

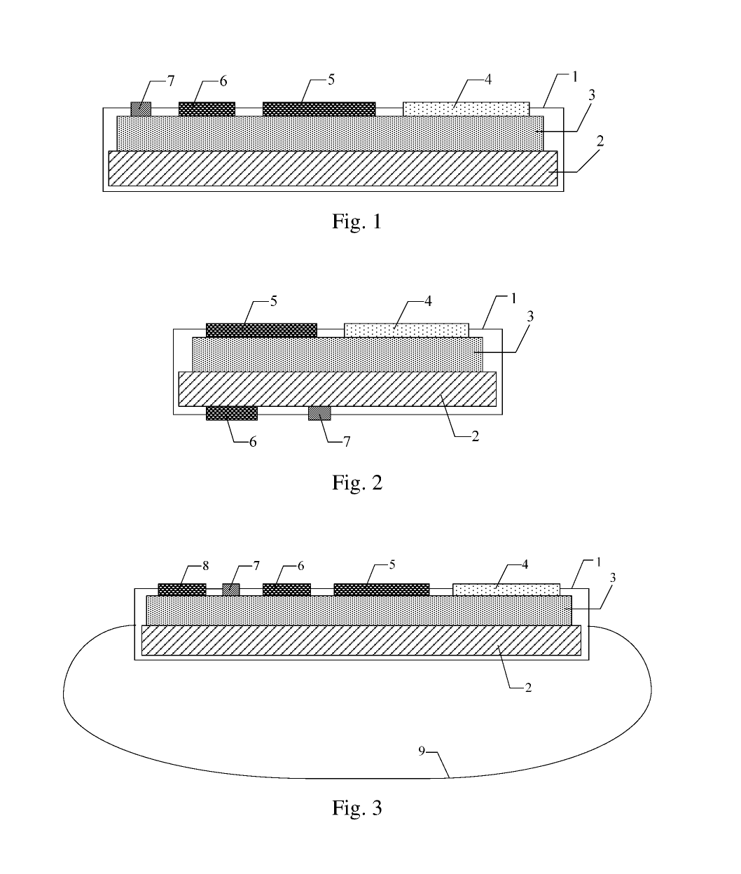

As illustrated in FIGS. 1-4, a sphygmomanometer as provided by embodiments of the present invention comprises a housing 1; a circuit board 2 and a power supply 3 that are disposed within the housing 1; a display 4 embedded at a surface of the housing 1; and a first electrode 5, a second electrode 6 and a photoelectric sensor 7 that are electrically connected to the circuit board 2, respectively; wherein the first electrode 5, the second electrode 6 and the photoelectric sensor 7 are embedded at a surface of the housing 1.

During practical operation of the above-mentioned sphygmomanometer as provided by the embodiment of the present invention, for measuring the blood pressure, it only requires a user to contact the first electrode and the second electrode with his/her two hands respectively while contacting the photoelectric sensor with his/her one hand; as compared with the existing sphygmomanometer, it's simpler in operation without the need of wearing a wrist strap belt; moreover, the first electrode, the second electrode and the photoelectric sensor are all embedded at the surface of the housing so that it not only doesn't affect the acquisition of electrocardiosignal of the first electrode and the second electrode and the acquisition of pulse wave signal of the photoelectric sensor but also achieves miniaturization and portability of the sphygmomanometer by integrating the first electrode, the second electrode and the photoelectric sensor within the housing, thereby satisfying the demand of user who requires for measurement of blood pressure whenever and wherever possible.

In particular implementation, for the above-mentioned sphygmomanometer as provided by the embodiment of the present invention, a processer and an amplifier that are electrically connected can be integrated on the circuit board. Particularly, the first electrode and the second electrode are electrically connected to two ends (a positive end and a negative end) of the amplifier respectively; the photoelectric sensor is electrically connected to the processer; the power supply is electrically connected to the processer, the amplifier, the photoelectric sensor and the display for supplying electrical power to the processer, the amplifier, the photoelectric sensor and the display (and/or Bluetooth).

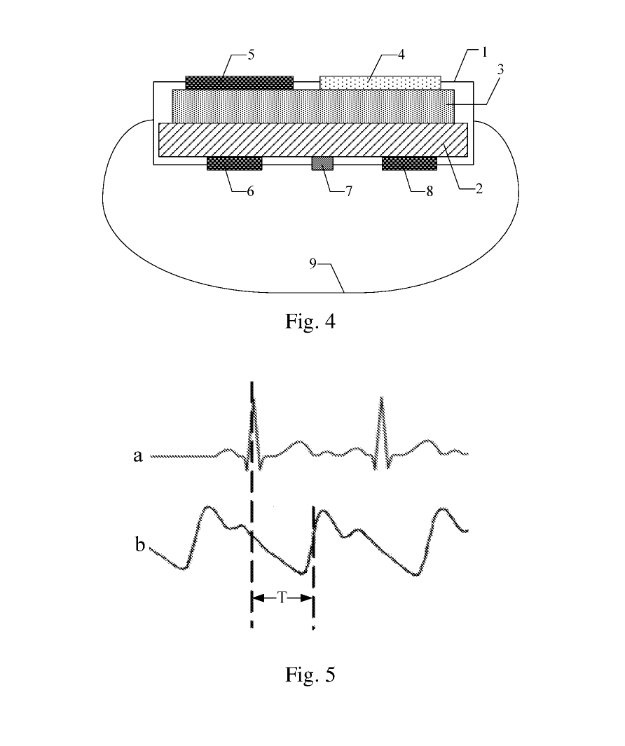

Hereinafter, particular working principle of the above-mentioned sphygmomanometer as provided by the embodiment of the present invention will be described in more details. The user contact the first electrode and the second electrode respectively with his/her two hands while contacting the photoelectric sensor with his/her one hand for a certain period of time (usually 10 seconds), so as to measure the blood pressure. During such operation, the amplifier is configured to acquire electrocardiosignal through the first electrode and the second electrode under a control of the processer and send the electrocardiosignal as acquired to the processer; the photoelectric sensor is configured to acquire pulse wave signal under a control of the processer and sends the pulse wave signal as acquired to the processer; the processer is configured to receive the electrocardiosignal sent by the amplifier and the pulse wave signal sent by the photoelectric sensor, recognize a peak point of the electrocardiosignal and a peak point of the pulse wave signal, determine a time difference between the peak point of the electrocardiosignal (as indicated by a curve a illustrated in FIG. 5) and the peak point of the pulse wave signal (as indicated by a curve b illustrated in FIG. 5) as a pulse transmission time (as indicated by T as illustrated in FIG. 5), substitute the pulse transmission time into an equation of blood pressure versus pulse transmission time as pre-stored to calculate a blood pressure value, and send the blood pressure value to the display; the display receives and displays the blood pressure value sent by the processer.

It should be explained that, in the above-mentioned sphygmomanometer as provided by the embodiment of the present invention, the equation of blood pressure versus pulse transmission time pre-stored in the processor can be obtained by way of calibration. In particular, measuring a standard blood pressure of a human body by using a standard mercury sphygmomanometer and measuring a current pulse transmission time of the human body by using the sphygmomanometer to be calibrated at the same time, so as to obtain a group of data of blood pressure versus pulse transmission time; measuring a plurality of groups of data of blood pressure versus pulse transmission time and linearly fitting the plurality of groups of data, so as to obtain the equation of blood pressure versus pulse transmission time for the sphygmomanometer to be calibrated; then inputting the equation into the processer.

During particular implementation, when a user measures blood pressure by using the above-mentioned sphygmomanometer as provided by the embodiment of the present invention, he/she contacts both the first electrode and the second electrode with his/her two hands respectively while contacting the photoelectric sensor with his/her one hand. Particularly, the user can choose to contact the first electrode and the photoelectric sensor with the same hand, and to contact the second electrode with the other hand; or, the user can choose to contact the second electrode and the photoelectric sensor with the same hand, and to contact the first electrode with the other hand; however, embodiments of the present invention are not limited thereto. Hereinafter the case where the user contacts the second electrode and the photoelectric sensor with the same hand and contacts the first electrode with the other hand will be described by way of example.

In some embodiments, for the above-mentioned sphygmomanometer as provided by the embodiment of the present invention, in order for the convenience of the user to contact both the second electrode and the photoelectric sensor at the same time with the same hand, as illustrated in FIGS. 1-4, the second electrode 6 and the photoelectric sensor 7 can be embedded at the same side of the housing 1, that is, a surface of the second electrode 6 and a surface of the photoelectric sensor 7 are exposed from the same side of the housing 1.

In some embodiments, for the above-mentioned sphygmomanometer as provided by the embodiment of the present invention, when the second electrode and the photoelectric sensor are embedded at the same side of the housing, as illustrated in FIG. 1, the first electrode 5 and the second electrode 6 can be embedded at the same side of the housing 1; in this way, the first electrode 5, the second electrode 6 and the photoelectric sensor 7 are all embedded at the same side of the housing 1, that is, a surface of the first electrode 5, a surface of the second electrode 6 and a surface of the photoelectric sensor 7 are all exposed from the same side of the housing 1; in this way, when measuring a blood pressure by using the above-mentioned sphygmomanometer, the user can press one of his/her hands on the first electrode 5 while pressing the other hand on both the second electrode 6 and the photoelectric sensor 7 for a certain period of time so that the display 4 of the sphygmomanometer can display a blood pressure value. At this time, in order for the convenience of the user to view the blood pressure value displayed on the display 4, as illustrated in FIG. 1, the display 4 can also be embedded at the side so that all of the first electrode 5, the second electrode 6, the photoelectric sensor 7 and the display 4 can be embedded at the same side of the housing 1, that is, a surface of the first electrode 5, a surface of the second electrode 6, a surface of the photoelectric sensor 7 and a surface of the display 4 are all exposed from the same side of the housing 1.

Of course, the surface of the display can be exposed from any side of the housing; in particular, it can be adapted appropriately according to actual conditions by comprehensively considering several factors such as the convenience for the user to view the blood pressure value and the reduction of the size of the sphygmomanometer as far as possible; however, embodiments of the present invention are not limited thereto.

In some embodiments, for the above-mentioned sphygmomanometer as provided by the embodiment of the present invention, when the second electrode and the photoelectric sensor are embedded at the same side of the housing, as illustrated in FIG. 2, the first electrode 5 and the second electrode 6 can be embedded at two opposite sides of the housing 1 respectively, that is, a surface of the first electrode 5 and a surface of the second electrode 6 are exposed from two opposite sides of the housing 1 respectively; in this way, the size of the sphygmomanometer can be further reduced. At this time, in order for the convenience of the user to view the blood pressure value displayed by the display 4 and for the reduction of the size of the sphygmomanometer, as illustrated in FIG. 2, the first electrode 5 and the display 4 can be embedded at the same side of the housing 1, that is, a surface of the first electrode 5 and a surface of the display 4 both are exposed from the same side of the housing 1.

Of course, in case where the second electrode and the photoelectric sensor are embedded at the same side of the housing, the first electrode and the second electrode can also be embedded at two adjacent sides of the housing 1 respectively, that is, a surface of the first electrode and a surface of the second electrode are exposed from two adjacent sides of the housing respectively; however, embodiments of the present invention are not limited thereto.

In some embodiments, for the above-mentioned sphygmomanometer as provided by the embodiment of the present invention, in order to ensure that the user can fully contact the first electrode with his/her hand so as to guarantee an accuracy of the electrocardiosignal as acquired by the amplifier and also reduce the size of the sphygmomanometer as far as possible for achieving miniaturization and portability thereof, a length of the surface of the first electrode exposed from the housing can be controlled to be within a range from 15 mm to 25 mm, a width of the surface of the first electrode exposed from the housing can be controlled to be within a range from 8 mm to 12 mm, and a thickness of the first electrode can be controlled to be within a range from 1 mm to 1.5 mm.

In some embodiments, for the above-mentioned sphygmomanometer as provided by the embodiment of the present invention, in order to ensure that the user can fully contact the second electrode with his/her hand so as to guarantee an accuracy of the electrocardiosignal as acquired by the amplifier and also reduce the size of the sphygmomanometer as far as possible for achieving miniaturization and portability thereof, a length of the surface of the second electrode exposed from the housing can be controlled to be within a range from 15 mm to 25 mm, a width of the surface of the second electrode exposed from the housing can be controlled to be within a range from 8 mm to 12 mm, and a thickness of the second electrode can be controlled to be within a range from 1 mm to 1.5 mm.

In some embodiments, for the above-mentioned sphygmomanometer as provided by the embodiment of the present invention, in order to ensure that the user can fully contact the photoelectric sensor with his/her hand so as to guarantee an accuracy of the pulse wave signal as acquired by the photoelectric sensor and also reduce the size of the sphygmomanometer as far as possible for achieving miniaturization and portability thereof, a length of the surface of the photoelectric sensor exposed from the housing can be controlled to be within a range from 5 mm to 6 mm, a width of the surface of the photoelectric sensor exposed from the housing can be controlled to be within a range from 2 mm to 2.5 mm, and a thickness of the photoelectric sensor can be controlled to be within a range from 1 mm to 1.5 mm.

In some embodiments, as illustrated in FIG. 3 and FIG. 4, the above-mentioned sphygmomanometer as provided by the embodiment of the present invention can further comprise a third electrode 8 electrically connected to the circuit board 2; in particular, the third electrode 8 can be electrically connected to one end of the amplifier connected to the second electrode 6 so that the third electrode 8 can be used as a feedback electrode serving for suppressing common-mode interference, thereby improving an accuracy of the electrocardio signal as acquired by the amplifier; moreover, the third electrode 8 and the second electrode 6 are embedded at the same side of the housing 1, that is, a surface of the third electrode 8 and a surface of the second electrode 6 are exposed from the same side of the housing 1 so as to facilitate the user to contact the third electrode 8 and the second electrode 6 with one hand at the same time.

In some embodiments, for the above-mentioned sphygmomanometer as provided by the embodiment of the present invention, in order to ensure that the user can fully contact the third electrode with his/her hand so as to guarantee an accuracy of electrocardiosignal as acquired by the amplifier and also reduce the size of the sphygmomanometer as far as possible for achieving miniaturization and portability thereof, a length of the surface of the third electrode exposed from the housing can be controlled to be within a range from 15 mm to 25 mm, a width of the surface of the third electrode exposed from the housing can be controlled to be within a range from 8 mm to 12 mm, and a thickness of the third electrode can be controlled to be within a range from 1 mm to 1.5 mm.

In some embodiments, as illustrated in FIG. 3 and FIG. 4, the above-mentioned sphygmomanometer as provided by the embodiment of the present invention can further comprise a wrist strap belt 9 connected to the housing 1; in this way, the user can directly wear the sphygmomanometer on his/her wrist for convenience of measuring the blood pressure whenever and wherever possible. For example, when measuring the blood pressure with the sphygmomanometer as illustrated in FIG. 3, the user can take off the sphygmomanometer from the wrist, then presses one hand on the first electrode 5 while pressing the other hand on the second electrode 6, the photoelectric sensor 7 and the third electrode 8 for a certain period of time so that the display 4 can display a blood pressure value; when measuring the blood pressure with the sphygmomanometer as illustrated in FIG. 4, it's not necessary for the user to take off the sphygmomanometer from the wrist but only needs to directly press the hand without wearing the sphygmomanometer on the first electrode 5 and to allow the second electrode 5, the third electrode 8 and the photoelectric sensor 7 all contacting the wrist wearing the sphygmomanometer for a certain period of time so that the display 4 can display a blood pressure value; in this way, the operation of the sphygmomanometer is further simplified. It should be noted that, when wearing the sphygmomanometer as illustrated in FIG. 4, it has to allow the exposed surface of the second electrode 6, the exposed surface of the third electrode 8 and the exposed surface of the photoelectric sensor 7 all facing to the wrist of the user.

During particular implementation, for the above-mentioned sphygmomanometer as provided by the embodiment of the present invention, the processer can be an ARM processer capable of controlling an acquisition of signal (e.g., controlling a sampling rate and a signal magnification of the amplifier; controlling a sampling rate and an illumination intensity of the photoelectric sensor, etc.) and reading and processing data in real time.

During particular implementation, for the above-mentioned sphygmomanometer as provided by the embodiment of the present invention, the power supply can be a rechargeable battery, such as, a lithium battery.

It should be explained that, for the above-mentioned sphygmomanometer as provided by the embodiment of the present invention, the photoelectric sensor can preferably be a reflective photoelectric sensor. Because both of a luminotron and a photoreceiver of the reflective photoelectric are located at the same side of the user's hand, the reflective photoelectric sensor can be integrated within the housing with only the surface of the reflective photoelectric sensor being exposed, so as to ensure achieving miniaturization and portability of the sphygmomanometer.

It should be explained that the above-mentioned sphygmomanometer as provided by the embodiment of the present invention is not limited to a structure which displays the blood pressure value through a display, but also can utilize a Bluetooth in place of the display. In particular, the Bluetooth can send the blood pressure value calculated by the processer to a mobile phone or a computer of the user through which the blood pressure value can be read; in this way, the size of the sphygmomanometer can be further reduced so that the sphygmomanometer as provided by embodiments of the present invention is smaller and more portable.

Hereinafter particular dimensions of the above-mentioned sphygmomanometer as provided by embodiments of the present invention will be described in more details with reference to the sphygmomanometer as illustrated in FIG. 4 by way of example. According to particular dimensions of the first electrode, the second electrode, the photoelectric sensor and the third electrode, by considering that a thickness of a circuit board and a lithium battery usually are 3 mm and 4 mm respectively, it can be deduced that the above-mentioned sphygmomanometer as provided by embodiments of the present invention has a length of 35 mm to 45 mm, a width of 28 mm to 35 mm and a thickness of 9 mm to 15 mm; more particularly, the length is about 40 mm, the width is about 30 mm, and the thickness is about 10 mm. The above-mentioned sphygmomanometer as provided by embodiments of the present invention is small in size and convenient for portability.

Embodiments of the present invention provide a sphygmomanometer, during practical operation, in order to measure the blood pressure, it only requires a user to contact the first electrode and the second electrode with his/her two hands respectively while contacting the photoelectric sensor with his/her one hand; as compared with the existing sphygmomanometer, it's simpler in operation without the need of wearing a wrist strap belt; moreover, the first electrode, the second electrode and the photoelectric sensor are all embedded at the surface of the housing so that it not only doesn't affect the acquisition of electrocardiosignal of the first electrode and the second electrode and the acquisition of pulse wave signal of the photoelectric sensor but also achieves miniaturization and portability of the sphygmomanometer by integrating the first electrode, the second electrode and the photoelectric sensor within the housing, thereby satisfying the demand of user who requires for measurement of blood pressure whenever and wherever possible.

The foregoing are merely exemplary embodiments of the present invention but not to limit the present invention thereto. The scope of protection of the present invention shall be defined by the appended claims.

The present application claims priority of Chinese Patent Application No. 201510275205.5 filed on May 26, 2015, the disclosure of which is incorporated herein by reference in its entirety as part of the present application.

1. A sphygmomanometer, comprising:
a housing;
a circuit board disposed within the housing; and
a first electrode, a second electrode and a photoelectric sensor that are electrically connected to the circuit board, respectively;
wherein,
the first electrode, the second electrode and the photoelectric sensor are provided at a surface of the housing.

2. The sphygmomanometer of claim 1, wherein the second electrode and the photoelectric sensor are provided at a same side of the housing.

3. The sphygmomanometer of claim 1, wherein the first electrode and the second electrode are provided at a same side of the housing.

4. The sphygmomanometer of claim 2, wherein the first electrode and the second electrode are provided at two opposite sides of the housing, respectively.

5. The sphygmomanometer of claim 1, wherein a surface of the first electrode exposed from the housing has a length of 15 mm to 25 mm, the surface of the first electrode exposed from the housing has a width of 8 mm to 12 mm, and the first electrode has a thickness of 1 mm to 1.5 mm.

6. The sphygmomanometer of claim 1, wherein a surface of the second electrode exposed from the housing has a length of 15 mm to 25 mm, the surface of the second electrode exposed from the housing has a width of 8 mm to 12 mm, and the second electrode has a thickness of 1 mm to 1.5 mm.

7. The sphygmomanometer of claim 1, wherein a surface of the photoelectric sensor exposed from the housing has a length of 5 mm to 6 mm, the surface of the photoelectric sensor exposed from the housing has a width of 2 mm to 2.5 mm, and the photoelectric sensor has a thickness of 1 mm to 1.5 mm.

8. The sphygmomanometer of claim 1, further comprising a third electrode electrically connected to the circuit board;
wherein the third electrode and the second electrode are provided at a same side of the housing.

9. The sphygmomanometer of claim 8, wherein a surface of the third electrode exposed from the housing has a length of 15 mm to 25 mm, the surface of the third electrode exposed from the housing has a width of 8 mm to 12 mm, and the third electrode has a thickness of 1 mm to 1.5 mm.

10. The sphygmomanometer of claim 1, further comprising a wrist strap belt connected to the housing.

11. The sphygmomanometer of claim 1, further comprising a display provided at the surface of the housing or a Bluetooth device.

12. The sphygmomanometer of claim 11, further comprising a processer and an amplifier which are integrated on the circuit board,
wherein the first electrode and the second electrode are electrically connected to a positive end and a negative end of the amplifier, respectively;
the photoelectric sensor is electrically connected to the processer.

13. The sphygmomanometer of claim 12, wherein the amplifier is configured to acquire electrocardiosignal through the first electrode and the second electrode under a control of the processer and send the electrocardiosignal as acquired to the processer; the photoelectric sensor is configured to acquire pulse wave signal under a control of the processer and sends the pulse wave signal as acquired to the processer; the processer is configured to receive the electrocardiosignal sent by the amplifier and the pulse wave signal sent by the photoelectric sensor, recognize a peak point of the electrocardiosignal and a peak point of the pulse wave signal, determine a time difference between the peak point of the electrocardiosignal and the peak point of the pulse wave signal as a pulse transmission time, substitute the pulse transmission time into the equation of blood pressure versus pulse transmission time to calculate a blood pressure value.

14. The sphygmomanometer of claim 1, wherein the sphygmomanometer comprise no wrist strap belt.

15. The sphygmomanometer of claim 1, wherein the sphygmomanometer has a length of 35 mm to 45 mm, a width of 28 mm to 35 mm, and a thickness of 9 mm to 15 mm.

16. The sphygmomanometer of claim 11, wherein the Bluetooth device is configured to be signal connected to an external receiving device.

17. The sphygmomanometer of claim 16, wherein the external receiving device is at least one selected from the group consisting of a mobile phone and a computer.

18. The sphygmomanometer of claim 12, wherein the processer is configured to store an equation of a blood pressure versus a pulse transmission time, the equation reflecting a relationship between the blood pressure and the pulse transmission time.

19. The sphygmomanometer of claim 1, further comprising a power supply disposed within the housing.
